(19) United States
(12) Patent Application Publication
(10) Pub. No.: US 2023/0173143 A1
(43) Pub. Date: Jun. 8, 2023

(54) MARKING MADE OF POROUS MATERIAL

(71) Applicant: Heraeus Deutschland GmbH & Co. KG, Hanau (DE)

(72) Inventor: Oliver KEITEL, Hanau (DE)

(21) Appl. No.: 18/058,551

(22) Filed: Nov. 23, 2022

(30) Foreign Application Priority Data
Dec. 2, 2021 (DE) 102021131770.3

(51) Int. Cl. A61L 31/02; A61L 29/02

(52) U.S. Cl. CPC A61L 31/022; A61L 29/02; A61L 2420/02

(57) ABSTRACT

The present invention relates to a medical instrument which comprises a porous metal layer. Also described are methods for producing such a medical instrument. The porous metal layer can serve as a marking for use in imaging radiological methods such as, for example, x-ray or ultrasound images.

### CROSS-REFERENCE TO RELATED APPLICATIONS

This application claims priority pursuant to 35 U.S.C. 119(a) to German Application No. 102021131770.3, filed Dec. 2, 2021, which application is incorporated herein by reference in its entirety.

### FIELD OF THE INVENTION

The invention relates to medical instruments having a marking for use in connection with imaging methods in surgical operations.

### TECHNICAL BACKGROUND

Medical instruments such as, for example, catheters, guiding sheaths, needles and medical electrodes are frequently provided with markings in order to allow better tracking of the positioning of the instrument by imaging methods such as, for example, x-ray or ultrasound images during use in a patient. For example, electrodeposition can be used to apply a radiopaque gold layer to a steel needle. In the case of instruments made of Nitinol to which such gold layers have reduced adhesion, additional rings or rivets made of a radiopaque material are often attached to the instruments instead.

Polymers having enclosed gas bubbles are frequently used as markings with ultrasonically active properties, as described, for example, in U.S. Pat. No. 6,106,473A. In addition, polymers having embedded particles made of radiopaque materials such as iron oxide or barium sulfate are used as markings. Until now, different types of markings, which have either radiopaque or ultrasound-echogenic properties, but not both, have usually been used. In addition, the production of such markings is in some cases complex and not very flexible. Problems with the stability of the marking can result from the application of a metal part that is only mechanically attached or from a coating with a soft plastics material. Mechanically fastened metal parts may have to be encapsulated to prevent sharp edges and stability problems.

### Preferred Embodiments

The object of the present invention is to solve one or more of the problems described above and further problems of the prior art. For example, the invention enables the provision of medical instruments having markings which have radiopaque or ultrasound-echogenic properties. The markings can have particularly good compatibility and biocompatibility. The invention further provides methods for producing such markings that are particularly easy to carry out and allow great flexibility with respect to the products which can be produced therewith. The markings can be applied to different materials.

These objects are achieved by the methods and devices described herein, in particular those that are described in the claims.

Preferred embodiments of the invention are described below.

[1.] A medical instrument characterized in that it comprises a porous metal layer.

[2.] The medical instrument according to Embodiment 1, wherein the metal layer comprises or consists of gold.

[3.] The medical instrument according to either of the preceding embodiments, wherein the metal layer is applied to a region of the medical instrument, wherein the region comprises a metal or an alloy selected from the group consisting of Pt, 1r, Ta, Pd, Ti, Fe, Au, Mo, Nb, W, Ni, Ti, MP35N, 316L, 301, 304 and Nitinol, wherein the alloy is preferably Nitinol.

[4.] The medical instrument according to Embodiment 3, wherein the region (102) is a metal carrier foil applied to the medical instrument.

[5.] The medical instrument according to Embodiment 4, wherein the porous metal layer covers a surface of the metal carrier foil only incompletely.

[6.] The medical instrument according to any one of the preceding embodiments, wherein the metal layer has radiopaque and/or ultrasound-echogenic properties.

[7.] The medical instrument according to any one of the preceding embodiments, comprising a catheter, a guiding sheath, a needle or a medical electrode.

[8.] The medical instrument according to any one of the preceding embodiments, wherein the metal layer comprises non-metallic particles, wherein the non-metallic particles preferably comprise a ceramic material, more preferably glass.

[9.] The medical instrument according to Embodiment 8, wherein the non-metallic particles have an average diameter of 100 nm to 100 μm, preferably 30 μm to 50 μm.

[10.] The medical instrument according to Embodiments 8 or 9, wherein the non-metallic particles have the shape of hollow bodies, preferably hollow spheres.

[11.] The medical instrument according to any one of the preceding embodiments, wherein the metal layer has a layer thickness of 5 μm to 250 μm, preferably 50 μm to 150 μm.

[12.] The medical instrument according to any one of the preceding embodiments, wherein the metal layer is a sintered metal layer.

[13.] The medical instrument according to any one of the preceding embodiments, wherein the metal layer has an open or closed porosity.

[14.] A method for coating a medical instrument, comprising the following steps:
(a) coating a region of a medical instrument with a composition, wherein the composition comprises metal particles and a carrier substance,
(b) heating the region and the composition in order to convert the composition into a porous metal layer, and
(c) thereby obtaining a medical instrument coated with a porous metal layer.

[15.] The method according to Embodiment 14, wherein the composition comprises gold.

[16.] The method according to Embodiments 14 or 15, wherein the heating of the region (102) and the composition is carried out at an oven temperature of 500° C. to 600° C.

[17.] The method according to any one of Embodiments 14 to 16, wherein the surface to be coated of the region (102) is roughened prior to being coated with the composition.

[18.] The method according to any one of Embodiments 14 to 17, wherein the composition is applied to the region (102) using screen printing, ink-jet printing or metered pasting.

[19.] The method according to any one of Embodiments 14 to 18, wherein the composition is applied to the region (102) in the shape of a prespecified pattern.

[20.] The method according to any one of Embodiments 14 to 19, wherein the region (102) is a metal carrier foil which is optionally connected to the remaining part of the medical instrument only after the formation of the porous metal layer.

### DETAILED DESCRIPTION

In principle, for the embodiments described herein, the elements of which “contain” or “comprise” a particular feature (e.g., a material), a further embodiment is always considered in which the element in question consists of that feature alone, i.e., comprises no further components. The word “comprise” or “comprising” is used herein synonymously with the word “contain” or “containing”.

If an element is referred to in the singular in an embodiment, an embodiment is also being considered in which several of these elements are present. The use of a term for an element in the plural fundamentally also encompasses an embodiment in which only a single corresponding element is contained.

Unless otherwise indicated or clearly precluded from the context, it is possible in principle, and is herewith clearly taken into consideration, that features of different embodiments may also be present in the other embodiments described herein. It is also contemplated in principle that all features that are described herein in conjunction with a method are also applicable to the products and devices described herein, and vice versa. Only for reasons of succinct presentation are all such contemplated combinations not explicitly listed in all instances. Technical solutions which are known to be equivalent to the features described herein are also intended to be encompassed in principle by the scope of the invention.

A first aspect of the invention relates to a medical instrument which comprises a porous metal layer. Medical instruments according to the invention described herein preferably comprise tools which are provided for surgical procedures on the human or animal body. In many cases, such tools contain a base body made of metal, or at least a section that comprises a metal surface. According to the invention, the medical instrument is equipped with a porous metal layer. This porous metal layer is preferably arranged on a surface of the instrument which is intended to be introduced into the human or animal body during a surgical intervention. The porous metal layer can preferably be configured as a marking which enables the attending physician to position the medical instrument in the body using an imaging method. Examples of such an imaging method are x-ray and ultrasound video methods.

The metal layer can comprise a radiopaque material, for example a metal, in particular a noble metal. Noble metals can be understood to be metals, the redox pairs of which have a positive standard potential with respect to the normal hydrogen electrode. A particularly preferred noble metal is gold. In one embodiment, the porous metal layer comprises gold or consists of gold.

The metal layer can be applied to a region of the medical instrument that comprises a metal or an alloy. This means that a metallic surface of the medical instrument is directly or indirectly covered with the porous metal layer. Examples of suitable metals are Pt, Jr, Ta, Pd, Ti, Fe, Au, Mo, Nb, W, Ni and Ti. Examples of suitable alloys are MP35N, 316L, 301, 304 and Nitinol.

In one embodiment, said region of the medical instrument comprises a non-noble metal or a stainless steel alloy. Examples of stainless steel alloys are 316L, 301 and 304.

MP35N is a nickel-cobalt-based hardenable alloy. A variant of MP35N is described in industry standard ASTM F562-13. In one embodiment, MP35N is an alloy comprising 33 to 37% Co, 19 to 21% Cr, 9 to 11% Mo, and 33 to 37% Ni.

PtIr10 is an alloy made of 88 to 92% platinum and 8 to 12% iridium.

PtIr20 is an alloy of 78 to 82% platinum and 18 to 22% iridium.

316L is an acid-resistant CrNiMo austenitic steel with approx. 17% Cr; approx. 12% Ni, and at least 2.0% Mo. A variant of 316L is described in industry standard 10088-2. In one embodiment, 316L is an alloy comprising 16.5 to 18.5% Cr; 2 to 2.5% Mo, and 10 to 13% Ni.

301 is a chromium-nickel steel with high corrosion resistance. A variant of 301 is described in industry standard DIN 1.4310. In one embodiment, 301 is an alloy comprising 16 to 18% Cr and 6 to 8% Ni.

Nitinol is a shape-memory nickel-titanium alloy having an ordered cubic crystal structure and a nickel content of approximately 55%, the remaining portion consisting of titanium. Nitinol has good biocompatibility and corrosion-resistance properties.

Unless otherwise indicated, all percentages given herein are to be understood as weight percent (wt. %).

In some embodiments, it may be desirable to fasten the porous metal layer to the medical instrument using a metal carrier foil. In this case, said region of the medical instrument is therefore formed by a metal carrier foil.

Accordingly, one embodiment of the invention is a medical instrument, wherein the region is a metal carrier foil applied to the medical instrument. The porous metal layer is applied directly or indirectly, for example via an adhesion promoter, to the metal carrier foil. The porous metal layer is preferably applied directly to the metal carrier foil.

The metal carrier foil can be completely or only incompletely covered by the porous metal layer on a surface of the metal carrier foil. In some cases, it may be desirable for the metal layer to be applied in the shape of a prespecified pattern, wherein this shape of the pattern differs from the shape of the metal carrier foil. In such an embodiment, the porous metal layer therefore only incompletely covers the surface of the metal carrier foil, i.e., a part of the metal carrier foil remains uncovered and freely accessible from the outside, even after the metal carrier foil has been fastened to the medical instrument.

In some embodiments, the medical instrument comprises a region comprising a polymer. In one embodiment, the porous metal layer is arranged on the polymer. Examples of suitable polymers are PET, ETFE, PTFE, FEP, PFA, PU, PI, PEEK, PVDF, a polyolefin, a silicone or an elastomer.

The porous metal layer can have radiopaque or ultrasound-echogenic properties, or have both radiopaque and ultrasound-echogenic properties. This means that during a surgical procedure by the attendant physician, the porous metal layer provides sufficient contrast in the x-ray or ultrasound image to enable clear determination of the position of the medical instrument in the body of the subject to be treated during the procedure.

The medical instrument can comprise, for example, a catheter, a guiding sheath, a needle or a medical electrode. Such a medical electrode can be configured to deliver an electrical signal to the human or animal body or to receive an electrical signal from the human or animal body. The medical instrument can be, for example, a lead, pulse generator, cardiac pacemaker, cardiac resynchronization device, sensor or stimulator. Leads are electrical wires which can be used, for example, in medical applications such as neuromodulation, cardiac stimulation, deep-brain stimulation, spinal-cord stimulation, or gastric stimulation. In one embodiment, the lead is configured and/or intended to be connected to a generator of an active implantable device. A lead can also be used in a medical device to receive an electrical signal. A stimulator is a medical device which can achieve a physiological effect by sending an electrical signal to the body of a living being. For example, a neurostimulator may produce an electrical signal in the nerve cell (e.g. an action potential) by delivering an electrical signal to a nerve cell.

In one embodiment, the metal layer comprises non-metallic particles. Such particles can improve the ultrasound-echogenic properties of the porous metal layer. Porous metal layer is understood to mean that the metal layer does not form a completely filled-out body made of a homogeneous material, but rather contains inclusions and/or cavities which are filled either with air or with a solid material. It is also possible that the porous metal layer comprises both air-filled cavities and inclusions in the form of incorporated particles. Such particles can consist of a non-metallic material, for example of a ceramic material. A preferred example of such a ceramic material is glass. Glass is a ceramic material which consists substantially of silicon dioxide (SiO₂). The non-metallic particles can have, for example, an average diameter of 100 nm to 100 μm, preferably 30 μm to 50 μm. In one embodiment, the non-metallic particles have a diameter of approximately 20 μm to approximately 50 μm. The non-metallic particles can have the shape of hollow bodies, for example the shape of hollow spheres. In one embodiment, the non-metallic particles are hollow spheres made of glass. Such hollow glass spheres are commercially available for example from 3M.

In one embodiment, the metal layer has a layer thickness of 5 μm to 250 μm, preferably 50 μm to 150 μm. In one embodiment, the layer thickness of the metal layer is approximately 80 μm to approximately 120 μm.

In some embodiments, the metal layer is a sintered metal layer. This means that the metal layer is formed from individual particles which are connected to one another at their points of contact, but are not fused completely to form a compact mass. Due to their structure such sintered structures can be differentiated, without particular effort by a person skilled in the art, from porous metal layers which are produced in another way, since the original particles are generally still recognizable after sintering. By contrast, metal layers produced by electrodeposition, for example, frequently have tree-like branched structures, as shown, for example, in US2015316499A1.

The porous metal layer can have an open or closed porosity. In this context, “open porosity” is understood to mean that the individual pores within the porous metal layer are predominantly connected to other pores, while, in the case of a “closed porosity”, the individual pores within the porous metal layers are predominantly not connected to one another, but are completely separated from one another by solid material.

In the context of this invention, pores are understood to mean micropores, mesopores and macropores. Micropores have a pore size in the range of less than 2 nm, mesopores have a pore size in the range of 2 to 50 nm and macropores have a pore size in the range of 50 to 5000 nm.

The pore size is preferably understood to mean the average size of the pores of the porous material. Accordingly, pore volume is preferably understood to mean the sum of the volumes of such pores.

In a preferred embodiment, the porous metal layer comprises micropores. The maximum of the pore diameter distribution particularly preferably lies within the range of the micropores.

The maximum of the pore diameter distribution particularly preferably lies within the range of 0.1 to 20 μm, preferably in the range of 1 to 10 μm, preferably also within a range of 0.5 to 10 μm or particularly preferably a range of 0.5 to 3 μm.

The porous metal layer can have a porosity of, for example, 1 to 60%, preferably 5 to 40%.

The porosity of a sample represents the ratio of the voids volume of the sample and total volume of the sample. For example, a sample with a total volume of 1 mm³ and a voids volume of 0.15 mm³ has a porosity of 15%. The percentile specification of porosity is an indication in volume percent (vol %).

To measure the porosity, metallographic specimens can firstly be produced by embedding in epoxy resin, grinding with SiC paper having a successively smaller grit size, and polishing with a diamond paste. In the following, images of the sample surface thus treated can be taken with an electron microscope (for example, Zeiss Ultra 55, Carl Zeiss AG). An optimally high contrast should hereby be achieved between the pores of the sample and the material (metal and ceramic). To evaluate the images, these gray scale images can be converted into binary images using the Otsu method. This means that the image pixels are in each case assigned to a pore or the sample material by means of a threshold value. The porosity is then determined on the basis of the binary images as a quotient from the number of pixels representing pores and the total number of pixels per image. Here, the porosity can be determined as an arithmetical mean of 5 images, in each case recorded from 5 polished specimens.

The invention further relates to a method for coating a medical instrument, comprising the following steps:
(a) coating a region of a medical instrument with a composition, wherein the composition comprises metal particles and a carrier substance,
(b) heating the region and the composition in order to convert the composition into a porous metal layer, and
(c) thereby obtaining a medical instrument coated with a porous metal layer.

The composition can be a metal paste or a suspension. The composition comprises metal particles and a carrier substance. The metal particles are preferably dispersed in the carrier substance. Examples of suitable carrier substances are water and organic solvents. Organic solvents suitable for the present invention are generally known to a person skilled in the art. Preferred types of organic solvents suitable for the present invention are non-polar, polar aprotic or polar protic solvents, e.g., toluene, terpineol, texanol, isopropyl alcohol, ethyl acetate, or a combination of two or more thereof. Preferably, the carrier substance comprises a terpineol, for example alpha terpineol. The composition can contain a binder, for example glass frit, or can be free of such binders. “Binder” is understood herein to mean a substance which causes solidification when a metal paste is fired by sintering. If the composition is a metal paste, it can have a solids content of, for example, 85 to 95%. If the composition is a suspension, a correspondingly lower solids content can be used, for example less than 50%.

Unless otherwise indicated, all percentages given herein are to be understood as mass percent. Suitable metal pastes and suspensions are commercially available, or can be prepared by the methods customary in the art. One example of a suitable commercially available metal paste is the gold thick-film paste C 4350 from Heraeus.

The metal particles preferably consist of a biocompatible metal, for example a noble metal. A preferred noble metal is gold. In one embodiment, the composition therefore comprises gold in the form of metal particles.

The composition can be applied to the surface of the region of the medical instrument using any suitable method. For example, a metal paste can be applied to the region using screen printing or metered pasting. A liquid composition can be applied, for example, by means of ink-jet printing or a suitable liquid metering system, for example a microspotting method.

Depending on the type and nature of the region, the composition can be applied directly to the region, or firstly an adhesion promoter can be applied to the region before the region is coated with the composition. For example, a closed, i.e., non-porous, first metal layer can be applied to the region, and the composition can subsequently be applied to this first metal layer.

In some cases, it may be desirable for the medical instrument to be provided with a porous metal layer using an indirect method. For this purpose, the composition can first be applied to a carrier foil which is subsequently applied to the medical instrument. In one embodiment, the region provided with a porous metal layer is therefore a metal carrier foil which is connected to the remaining part of the medical instrument only after the formation of the porous metal layer.

In some embodiments, the medical instrument comprises a region comprising a polymer. In one embodiment, as described above, the composition is applied to a metal carrier foil and subsequently heated, as described herein, in order to form a porous metal layer thereon. The bond made of the metal carrier foil and porous metal layer obtained hereby can then be applied to the region comprising a polymer. Examples of suitable polymers include PET, ETFE, PTFE, FEP, PFA, PU, PI, PEEK, PVDF, a polyolefin, a silicone or an elastomer.

The grinding fineness of the metal paste can preferably be ≤20 μm, more preferably ≤15 μm. The grinding fineness of the paste can be determined according to DIN EN ISO 1524:2013-06.

After application, the composition is burnt onto the region in order to obtain a porous metal layer. This is done by heating the region and the composition in order to convert the composition into a porous metal layer. The region and the composition are preferably heated at an oven temperature of less than 800° C., for example 500° C. to 600° C., or at less than 500° C. In order to improve the adhesion of the porous metal layer to the region, the surface of the region can be roughened before the composition is applied. This can be done by mechanical methods or using a laser.

The present invention in principle allows a free design of the geometry of the porous metal layer. This can preferably be achieved by applying the composition to the region in the shape of a prespecified pattern. Using an ink-jet or screen-printing method, which in principle allows spatially resolved control of the application of a material, the compositions according to the invention can be printed onto the region in any shape so that a porous metal layer having the corresponding shape is formed at the end of the method. By selecting suitable shapes, it is possible, for example, to make it easier for the attendant physician to determine the position of the medical instrument in the body of a patient when the medical instrument is being used during a surgical procedure even better. Furthermore, it is possible to mark different instruments with different shapes.

### EXAMPLES

The invention is further illustrated below using examples which are, however, not to be understood as limiting. It will be apparent to the person skilled in the art that other equivalent means may be similarly used in place of the features described here.

### FIGURES

FIG. 1 shows a detail of a medical instrument 100 which comprises a region 102. The region 102 is designed to be provided with a porous metal layer.

FIG. 2 shows a schematic cross-sectional view of a region 102 covered with a composition 103. In this example, the composition 103 is a gold thick-film paste. By heating ΔT the region 102 together with the composition 103, a porous metal layer 101 forms on the region 102. The porous metal layer 101 comprises pores 104, wherein the individual pores 104 are connected to one another in some cases, and in other cases have singular structures separated from one another.

FIG. 3 shows a detail of a medical instrument 100 which is provided with a marking using a metal carrier foil 102 on which a porous metal layer 101 has been applied. In this example, the composition was applied to the metal carrier foil 102 in a prespecified pattern in order to obtain, by subsequent heating, a porous metal layer 101 having a corresponding shape. The metal carrier foil 102 with the porous metal layer 101 can then be fastened to the medical instrument 100, for example by gluing, soldering or welding.

FIG. 4 shows a metallographic detail of a ceramic having a porous gold layer in a cross-sectional view produced according to Example 4. The porous gold layer is shown in the center of the figure. It has a layer thickness of approximately 25 μm. The porous structure of the gold layer is clearly visible.

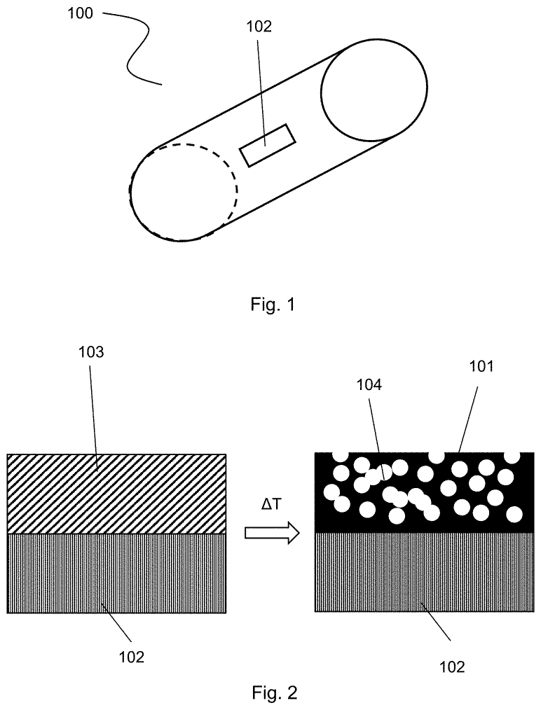

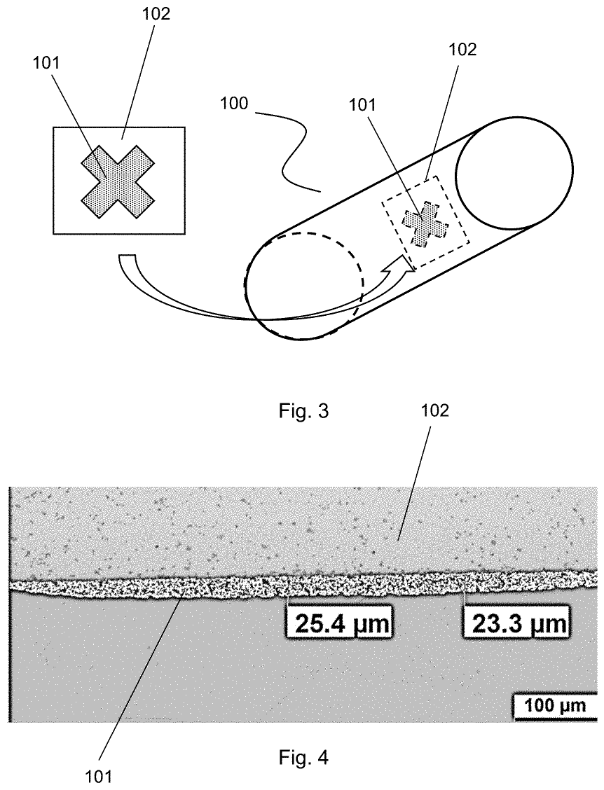

### Example 1: Applying a Porous Gold Layer to Steel

Using a metered pasting method, a gold thick-film paste (type C 4350, Heraeus Deutschland GmbH & Co KG, Hanau, Germany) was applied to a stainless steel foil in the form of strips having a width of 750 μm and a thickness of 40 μm. After firing at a temperature of 550° C., a porous gold layer with very good adhesion to the stainless steel foil was obtained.

### Example 2: Applying a Porous Gold Layer to Nitinol

According to Example 1, a porous gold layer was applied to a workpiece made of Nitinol. The adhesion of the gold layer was strong enough to withstand an adhesive tape test.

### Example 3: Applying a Porous Gold Layer to a Rough Surface

According to Example 2, a porous gold layer was applied to a workpiece made of Nitinol, of which the surface had previously been roughened. As a result, it was possible to improve the adhesion of the gold layer compared to the result in Example 2.

### Example 4: Applying a Porous Gold Layer to Ceramic

According to the method from Example 1, a porous gold layer having a thickness of 25 μm was applied to an aluminum oxide ceramic. The results are shown in FIG. 4.

What is claimed is:

1. A medical instrument, wherein it comprises a porous metal layer.

2. The medical instrument according to claim 1, wherein the metal layer comprises or consists of gold.

3. The medical instrument according to claim 1, wherein the metal layer is applied to a region (102) of the medical instrument, wherein the region comprises a metal or an alloy selected from the group consisting of Pt, Jr, Ta, Pd, Ti, Fe, Au, Mo, Nb, W, Ni, Ti, MP35N, 316L, 301, 304 and Nitinol, wherein the alloy is preferably Nitinol.

4. The medical instrument according to claim 3, wherein the region (102) is a metal carrier foil applied to the medical instrument.

5. The medical instrument according to claim 1, wherein the metal layer has radiopaque and/or ultrasound-echogenic properties.

6. The medical instrument according to claim 1, comprising a catheter, a guiding sheath, a needle or a medical electrode.

7. The medical instrument according to claim 1, wherein the metal layer comprises non-metallic particles, wherein the non-metallic particles preferably comprise a ceramic material, more preferably glass.

8. The medical instrument according to claim 1, wherein the metal layer has a layer thickness of 5 μm to 250 μm, preferably 50 μm to 150 μm.

9. A method for coating a medical instrument, comprising the following steps:
(a) coating a region (102) of a medical instrument with a composition (103), wherein the composition comprises metal particles and a carrier,
(b) heating the region (102) and the composition in order to convert the composition into a porous metal layer, and
(c) thereby obtaining a medical instrument coated with a porous metal layer.

10. The method according to claim 9, wherein the composition comprises gold.

11. The method according to claim 9, wherein the heating of the region and the composition is carried out at an oven temperature of 500° C. to 600° C.

12. The method according to claim 9, wherein the surface to be coated of the region is roughened prior to being coated with the composition.

13. The method according to claim 9, wherein the composition is applied to the region using screen printing, ink-jet printing or metered pasting.

14. The method according to claim 9, wherein the composition is applied to the region in the shape of a prespecified pattern.

15. The method according to claim 9, wherein the region is a metal carrier foil which is optionally connected to the remaining part of the medical instrument only after the formation of the porous metal layer.
